(19) United States
(12) Patent Application Publication
(10) Pub. No.: US 2009/0092687 A1
(43) Pub. Date: Apr. 9, 2009

(54) Prhormone composition and method of use thereof

(76) Inventor: Daniel S. Stein, Tierra Verde, FL (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/906,958

(22) Filed: Oct. 3, 2007

(51) Int. Cl. A61K 31/56; A61K 31/565; A61K 31/57; A61K 36/00; A61K 36/25; A61K 36/296; A61K 36/31; A61P 5/24

(52) U.S. Cl. 424/728; 424/725; 424/755; 514/170; 514/177; 514/178; 514/182

(57) ABSTRACT

The invention provides methods and compositions for maintaining a state of sexual wellness in a healthy human by providing a dietary supplement comprising one or more sexual steroid prohormones.

### FIELD OF THE INVENTION

The invention generally relates to compositions and methods for increasing sexual wellness in a healthy human subject.

### BACKGROUND OF THE INVENTION

Hormones are chemical messengers that transfer information via the bloodstream from one part of the body, generally an endocrine gland, to the body in general or to a specific target organ.

Target organs have specialized receptors that gather information that has been transferred from the circulatory system by hormones. An example of a target organ is the uterus, which is stimulated by the circulating hormone estrogen to develop uterine glands.

Prohormones are the building blocks of hormones and are not chemical messengers themselves. They are instead biochemical precursors of hormones, and while ineffective themselves in eliciting specific target organ responses, do nevertheless provide the building material from which hormone production can be naturally optimized by the body's own physiology.

Hormone production—for example, testosterone, estrogen, and progesterone—is regulated by a master gland called the pituitary.

Prohormones are not regulated by the pituitary. In general, the blood levels of sex steroid prohormones are not regulated by any substance. Instead, prohormones are generally available to assist in the production of hormones, which then act as chemical messengers to other target organs. Moreover, blood levels of sex steroid prohormones are not regulated by any substance, and they are generally available to assist in the production of the hormones themselves, which then act as chemical messengers to target organs.

### SUMMARY OF THE INVENTION

The present invention is based, in part, upon the discovery that prohormone compositions can increase the sexual wellness of healthy men and healthy women. Ingestion of the prohormone compositions results in improved, enhanced, enriched, rejuvenated sexual desire, sexual arousal, and sexual response in both healthy men and healthy women. While not wishing to be bound by theory it is believed that dietary supplementation with sex steroid prohormones results in an optimal natural synthesis of hormones supportive of enhanced sexual wellness when ingested by a healthy man or woman.

In one aspect, the invention provides a method of increasing sexual wellness in a healthy human subject by administering to the subject a non-toxic, effective amount of a composition comprising one or more sex steroid prohormones, or a pharmaceutically acceptable salt or metabolite thereof. In various embodiments, the prohormone is, e.g., dehydroepiandrosterone (DHEA), pregnenolone, androstenediol, or androstenedione.

In another aspect, the invention features a method of attaining and maintaining a desired healthy level of sexual wellness in a subject. The method includes assessing sexual wellness in a healthy subject and administering to the subject a non-toxic, effective amount of a composition comprising a prohormone, or a pharmaceutically acceptable salt or metabolite thereof. Sexual wellness following administration of said composition to the subject is evaluated. The composition is readministered to the subject as needed to maintain a desired level of sexual wellness.

In a further aspect, the invention provides a composition for enhancing sexual wellness in a subject. The composition can include one or more sex steroid prohormones in an amount effective to increase sexual wellness in a healthy human subject. Preferred prohormones include dehydroepiandrosterone (DHEA), pregnenolone, androstenedione and androstenediol.

In a still further aspect, the invention provides a method of making a pharmaceutical or nutriceutical composition for enhancing a level of sexual wellness in a subject by combining one or more sex steroid prohormones, e.g., dehydroepiandrosterone (DHEA), pregnenolone, androstenedione and androstenediol, in a pharmaceutical or nutriceutical composition an amount effective to increase sexual wellness when administered to a healthy human subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention provides a composition of one or more sex steroid prohormones that enhance sexual wellness when ingested by healthy men and women. Enhanced sexual wellness can manifest itself in increased sexual desire, sexual arousal, sexual performance and sexual response. While not wishing to be bound by theory, it is believed that administration of the steroid prohormone compositions of the invention promote optimum sex hormone levels or effects in healthy men and women to increase sexual wellness and global good health.

While prohormones have been reported to be useful previously for treating various types of subjects with sexual dysfunction, the present invention is based in part on the insight that administering one or more prohormones to healthy subjects can increase their sexual wellness, and accordingly, their overall health. Increased sexual wellness delays the aging process and can increase life span. In addition, increased sexual wellness is also associated with an increased quality of life, as it has been reported to enhance the immune system's ability to defend against bacteria and viruses, resist cancer, enhance the circulatory system, and ameliorate undesired stress-responses, particularly those associated with chronic stimulation of the adrenal gland (See, e.g., Stein, Passionate Sex: Discover the Special Power in You, Carroll & Graf Publishers, 2001, pp. 19-27; Lindau et al., N Engl. J Med. 357:762-74, 2007.

Prohormone Compositions

Prohormones suitable for inclusion in the compositions in the invention include any prohormone, or metabolite thereof, that produce increased sexual wellness when administered to a healthy subject. Preferred prohormones include sex steroid hormones, e.g., DHEA, pregnenolone, androstenedione, and androstenediol. These prohormones are available commercially with purity suitable for consumption to a healthy subject. Alternatively, they can be produced using synthetic methods known in the art.

The prohormone-containing compositions are preferably formulated and administered to a subject to induce desired sexual effects without also inducing undesired side effects, such as undesired anabolic or androgenic effects, in the subject.

In various embodiments, the composition is formulated specifically for administration to a male or female subject. For example, when formulated for administration to a male, suitable unit doses for DHEA are, e.g., 10 mg-200 mg, 12.5 mg to 150 mg, 15 mg-100 mg, 20 mg-75 mg, 50 mg, or 100 mg. When formulated for administration to a female, suitable unit doses are, e.g, 5 mg to 100 mg, 7.5 mg to 50 mg, 10 mg to 37.5 mg, 25 mg, 50 mg or 75 mg. Suitable unit doses for pregnenolone include are 10 mg to 2 grams for men, and 100 mg to 1 gram for women.

If desired, prohormone salts, esters, metabolites, and other derivatives of prohormones can be used in the composition, provided they are converted in vitro or in vivo to a form that acts to increase sexual wellness in a subject. Examples of modified prohormone forms include, e.g., DHEA sulfate (DHEAS) or an ester thereof, and the DHEA metabolite7-keto dehydroepiandrosterone (7-keto DHEA).

The composition may optionally include additional nutriceutical aphrodisiacs (sexual nutrients), herbal, vitamin, or mineral nutritional sex enhancement ingredients. Examples include, e.g, Yohimbe, Tribulus terrestris, Black Cohosh, Korean Siberian or other Ginseng, Maca, Pueraria Lobata Root, GABA, Wild yam extract, Muira puama extract, L-Arginine HCl, Vitamin B6, Eleutherococcus Extract, Xanthroparmelia scarbrosa, Velvet Deer Antler, Homey Goat Weed, Damiana, Pumpkin Seed, Stinging Nettle, Astragalus, Licorice Extract, Ho Shou Wu Extract, Hops, Zinc, and Boron; Damiana saffron, Tumeia aphrodisiaca, Irish moss, and fake unicorn root. If desired, the composition may additionally include one or more other vitamins or minerals.

The composition can be made by combining one or more sex steroid prohormones and optionally (a) and (b) additional dietary supplements such as vitamins and minerals, including zinc, which is reported to improve the endogenous testicular production of testosterone and Vitamin B, which is reported to promote vascular and neural health, this improving genital blood flow and neural sensitivity. The mixture is formed into a desired form for consumption, e.g., a unit dosage form and is optionally labeled as being useful for improving sexual health.

The prohormone composition may be consumed in any form that delivers an effective dose to a subject. In one embodiment, it is provided as a dietary supplement, e.g., a capsule, a tablet, a caplet, a liquid beverage, a powder beverage mix, a dietary gel, or as a ready-to-eat bar or drink product. The dosage form of a dietary supplement may be provided in accordance with customary processing techniques for herbal and/or dietary supplements, wherein the active ingredients are suitably processed and compressed into, e.g., tablets and/or caplets, with suitable excipients.

The dietary supplement may further comprise natural and/or artificial flavoring components, dyes or other coloring additives, preservatives and other conventional food supplement additives known in the art. If desired, the dietary supplement may further contain additional components to further increase the speed and or ease with which the substances enter the bloodstream. A dietary supplement form of the composition may be provided in accordance with customary processing techniques for herbal and/or dietary supplements in any of the forms mentioned above. For instance, in the embodiment in which the supplemental composition is provided is a capsule, the active ingredients may be suitably processed and encapsulated into capsules, e.g., cellulose, gelatin, etc., or compressed into caplets with cellulose, croscarmellose sodium, stearic acid, magnesium stearate, silica and other with suitable excipients. Those of skill in the art will appreciate that the supplemental composition may contain a variety of excipients.

Typically, a desired daily dosage is achieved by administering an appropriate quantity of unit dosages.

The prohormone composition may alternatively be formulated for alternative modes of delivery, e.g., for sublingual, transnasal, parenteral, subtopical transepithelial, as a transdermal patch, subdermal, or inhalation delivery. Preparation for delivery in a transdermal patch can be performed using methods also known in the art, including those described generally in, e.g., U.S. Pat. Nos. 5,186,938; 6,183,770; 4,861,800; 4,284,444 and WO 89/09051. Patches can be made to control the release of skin-permeable active ingredients over a 12 hour, 24 hour, 3 day, and 7 day period. The penetration through skin of specific formulations may be measures by standard methods in the art (for example, Franz et al., J. Invest. Derm. 64:194-195 (1975)).

The prohormone composition may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage may be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The composition can be provided as an article of manufacture in combination with labeling that the composition is to be taken for increasing sexual wellness subject. The article is typically a container or kit such as a bottle or box containing unit dosage forms (e.g., tablets, capsules) with labeling indicating the rate at which the unit dosage should be consumed.

Method of Administering a Prohormone Composition for Increasing Sexual Wellness

In general, a prohormone composition according to the invention is administered to a healthy subject in an amount sufficient to improve sexual wellness in a healthy man or woman.

As used herein a “healthy subject” is a human male or female that does not exhibit a physical or psychological impediment to engaging in sexual behavior. A “healthy subject” does not experience other sexual dysfunction secondary to disease (such as cancer, non-cancerous tumors, diabetes, or cardiovascular disease), or injury. As used herein, a “healthy woman” is does not have a sexual disorder, including hypoactive sexual desire disorder or women's sexual interest/desire disorder, subjective sexual arousal disorder, genital sexual arousal disorder, persistent sexual arousal syndrome, combined genital and subjective arousal disorder, dyspareunia, vaginismus and sexual aversion disorder. In some embodiments, a healthy woman is one that is not peri-menopausal and/or post-menopausal.

As used herein, a “healthy man” does not have a male sexual dysfunction such as aversion towards sex, low sexual desire, difficulty in achieving and in maintaining an erection, premature ejaculation, inhibited or delayed ejaculation, orgasm with flaccid penis, anorgasmia, pain at the time of coitus, dissatisfaction with frequency of intercourse per week, or dissatisfaction with partner, and/or dissatisfaction with one's own sexual function. Disorders are also discussed in, e.g. the Diagnostic Criteria for Research, ICD-10 Classification of Mental and Bevioural Disorders (World Health Organization, The ICD-10 classification of mental and behavioral disorders. Diagnostic criteria for research. World Health Organization: Geneva, 1993).

The healthy male or female subject does not have hypoactive desire, hypoactive arousal, orgasmic dysfunction, or mental illness sexual dysfunction. The healthy male subject additionally does not have erectile dysfunction or impotence.

The dietary supplement is typically administered in an amount effective to result in increased sexual wellness in a subject. The phrase “increased sexual wellness” means sexual wellness is increased in a subject following administration of the dietary supplement as compared to the level of sexual wellness experienced when the dietary supplement is not administered to the subject. As used herein, “an effective amount” refers to an amount effective for promoting sexual wellness when administered, e.g., as a serving one to three times a day over a period of time, e.g., several week(s), and most preferably for at least eight weeks. An effective amount of the supplemental dietary composition is typically from about 0.1 g to about 10 g of the supplemental dietary composition. More preferably, an effective amount of the composition comprises from about 1 mg to about 5 g of the supplemental dietary composition. Most preferably, an effective amount of the supplemental dietary composition comprises about 10 mg to 4.0 g of the nutritional composition.

Preferably, the composition does not produce androgen-associated side effects when administered to the subject. In their most common manifestations these may be oily skin, facial hair growth, hair loss, acne, cliteromegaly, deepening of voice, hirsuitism, and/or excessive libido for lifestyle. Preferably, the composition does not produce anabolic side effects when administered to the subject. In their most common manifestation there may be excessive muscle development relative to fat in response to isotonic resistance exercise.

In some embodiments, the administration of the prohormone composition of the invention increases testosterone levels when administered to a healthy subject. In other embodiments the invention increases testosterone effects, presumably by modifying testosterone receptor sensitivity or otherwise enhancing or modulating testosterone bioavailability or bioactivity.

Sexual wellness can be measured by one or more criteria typically used to assess sexual activity, including sexual function. Increased sexual wellness can be evaluated for any desired period of time. It can be assessed more specifically by evaluating, e.g., an increase in sexual desire, an increase in sexual pleasure, an increase in sexual passion, and/or improvement in the quality of orgasm, an increase in sexual fantasy, an actual increase in events of sexual frequency, an increase in sexual arousal, an increase in genital neurosensitivity, an increase in genital blood flow and associated lubrication, genital engorgement and swelling (clitoral and labial engorgement in women, ease of obtaining and/or maintaining penile erection and blood engorgement during erection (penile turgidity, firmness, size, or erection duration in men) or to improve satisfaction with overall sex life.

If desired, a prohormone composition of the invention is administered to effect one or more specific components of sexual wellness. The component can include, an increase in sexual desire, arousal, response, and performance, including frequency and fantasy, sexual pleasure, and sexual arousal. In women sexual arousal includes primarily lubrication and clitoral vulvar vascular engorgement and/or enhanced neurosensitivity. In men sexual arousal includes erectile strength, turgidity or size due to increased penile vascular engorgement and/or neurosensitivity. Other components of sexual wellness that can be measured include sexual response, including improved or heightened frequency or intensity of orgasm in sexual performance. In women, sexual performance is typically a reflection of enhanced desire and arousal. This is also the case in men, but with the addition of improved erection firmness and maintenance over time to allow for prolonged intimate contact.

An improvement in sexual wellness or component thereof can be objective or subjective and can include written assessments, including self-assessments, or measurement of one or more physiological indicia of sexual activity. Self assessments include those disclosed in the Examples, below, the Index of Female Sexual Function (IFSF). (See Urology 53: 481-486, 1999) and the International Index of Erectile Function (IIEF) questionnaire. (See Urology: 49, 822-830.)

Sexual wellness can also include improved ability to achieve an erection or an erection that can be maintained for a period of time that is longer than would occur without the administration of the dietary supplement. Sexual wellness can also be assessed by measuring an improvement in the quality of the climax or orgasm for the subject after ingesting a composition containing one or more prohormones. In a female, “climax” means orgasm whether by vulvar, vaginal, cervical or clitoral stimulation and can manifest itself as increased vaginal secretion, clitoral swelling, nipple erection, contraction of vaginal musculature and a general tingling sensation.

In general, the administration of the prohormone composition of this invention is carried out on a regular basis, e.g., daily basis, for a period of time sufficient to improve sexual wellness.

The time and amount administered will vary from subject to subject and will be influenced by the age of the subject, whether male or female. Generally, the younger the subject, the sooner the results will be seen and the smaller the amount needed to see the results. As a subject ages, the composition will have to be administered over a longer period of time and in larger amounts for a subject to experience results. The composition is preferably administered for at least three months. By ingesting the composition over an extended period of time making the dietary supplement an addition to the diet on a sustained basis, sexual wellness will be improved generally.

The invention will be illustrated in the following non-limiting examples.

### EXAMPLE 1

### Prohormone Formulation for a Male Subject

A tablet unit dosage form with the following ingredients is prepared (amounts are per serving)

| | | |
|---|---|---|
| Folate (Folic Acid) | 400 | mcg |
| Zinc (as Oxide) | 25 | mg |
| EXTENZE ™ Male Prohormone Support | | |
| Micronized DHEA | 50 | mg |
| Pregnenolone (3β-hydroxypren-5en-20-one) | 10 | mg |
| EXTENZE ™ Bio-Enhancement Support | | |
| Black Pepper (seed) | | |
| Piper Longum (seed) | | |
| Ginger (root) | | |
| EXTENZE ™ Sexual Response Support | 600 | mg |
| Yohimibe Extract (bark) (provides 12.5 mg | | |
| yohimbine alkaloids | | |
| Tribulus terrestris Extract (aerial part and fruit) provides | | |
| 50 mg furanosterols | | |
| Korean Ginseng Extract (root) Standardized to 10% | | |
| ginsenosides | | |
| Cnidiuim monnier (seed) | | |
| Eleutherococcus Extract Standardized to 0.8% elethrosides | | |
| Xanthroparmelia scarbrosa (aerial part) | | |
| Gamma-Aminobutyric Acid (GABA) | | |
| Velvet Deer Antler | | |
| Horny Goat Weed (leaf) | | |
| Damiana (leaf) | | |
| Muira puama Extract (stem) | | |
| Pumpkin (seed) | | |
| Stinging Nettle (root) | | |
| Astragalus (root) | | |
| Licorice Extract (root) | | |
| l-Arginine Hydrochloride | | |
| Ho Shou Wu Extract (root) | | |
| Hops (Strobile) Extract (blossom) | | |
| Boron (as Chelate) | | |

Inactive ingredients: Dicalcium phosphate, microcrystalline cellulose, Croscarmellose Sodium, Stearic Acid, Film Coating (Dextrin, Titanium Dioxide, Hydroxypropylmethyl Cellulose, FD& C Blue #1 Aluminum Lake, Macrogol/PEG 8000, Dextrose Monohydrate, Lecithin, Maltodextrine, Macrogol/PEG 400), Magnesium Stearate, Silica

### EXAMPLE 2

### Prohormone Formulation for a Female Subject

A tablet unit dosage form with the following ingredients is prepared (amounts are per serving)

| | | |
|---|---|---|
| Vitamin B6 (as pyridoxine HCl) | 25 | mg |
| Folate (as folic acid) | 400 | mcg |
| Boron (as boron chelate) | 250 | mcg |
| Yohimbe bark extract (providing 6.25 mg yohimbine | 78 | mg |
| alkaloids) | | |
| Black cohosh root standardized extract (2.5% triterpene | 40 | mg |
| glycosides as 27-deoxyactein) | | |
| DHEA | 25 | mg |
| Pregnenolone | 10 | mg |
| Tribulus terrestris extract (stem and dried fruit) | 111 | mg |
| (providing 50 mg saponins) | | |
| Korean ginseng root extract (10% ginsenosides) | 75 | mg |
| Eleuthero root extract (0.8% eleutherosides) | 50 | mg |
| GABA | 25 | mg |
| Deer antler | 5 | mg |
| Damiana (leaf) | 5 | mg |
| Muira puama extract (aerial parts) | 5 | mg |
| Astragalus root | 5 | mg |
| Licorice root extract | 5 | mg |
| L-Arginine HCl | 5 | mg |
| Ho-shou-wu extract (root) | 5 | mg |
| Bio-Enhancement Blend: | 25 | mg |
| Black pepper fruit, ginger root, and long pepper fruit | | |

Other ingredients: dicalcium phosphate, microcrystalline cellulose, croscarmellose sodium, stearic acid, magnesium stearate, silica and film coat (hypromellose, hydroxypropyl cellulose, and coloring agents)

### EXAMPLE 3

### Enhancement of Sexual Health in Healthy Subjects with a Prohormone-Containing Formulation

EXTENZE™ (ambesium labidrol) is medically designed formulation prepared as described in Example 1 with the objective of improving male sexual function. A limited initial pilot study was performed to obtain information of the effectiveness of the formulation and provide the basis for the design of future studies.

A group of 20 volunteers were given one tablet daily of a strategically, medically formulated dietary sexual nutritional supplement for eight weeks and a number of physical and perceptual assessments were performed. The process of participant selection started with advertisements in local papers. Specified criteria as to gender, age and sexual interest were integrated in the randomized selection development. This study was conducted in a private clinical medical setting. Based on the statically significant enhancement of sexual health, this formulation appears to be effective.

EXTENZE™ (aunctus philtrum), is a unique formulation that was medically designed from a variety of pro-hormones and pro-sexual nutrients that have been proclaimed to promote sexual health and well-being for decades and in some cases centuries. It was created as a non-prescription pro-sexual herbal and male pro-hormone supplement to enhance sexual health, penile size and erection and increase sexual desire. We hypothesized that in addition to promoting sexual health, penile erection, size, and sexual performance, the added sense of well-being would have an effect on desire for sex.

Since the market is flooded with herbal supplements claiming amazing results that are physically and physiologically impossible to achieve, it was our intent to document authentic, experienced results on sexual intimacy from a clinical perspective. The study objective was to determine if EXTENZE™ improves sexual functioning as defined by physical and perceptual self evaluations. The following are highlights of the rationale for medical inclusion of ingredients included in this product:

Tribute

Tribistol, the active ingredient in tribulus terrestris, has been demonstrated to increase the secretion of lutenizing hormone. Research suggests that the lutenizing hormone stimulates the release of testosterone, and LH enhances the level of testosterone production (Adaikan, Gauthaman, Prasad & Ng, 2000; Li, Shi, Xiong, Prassin, Tezuka, Hareyama, Wang, Tanaka, Namba, & Kadota, 1998). It has been used for centuries in Chinese medicine to treat a variety of aliments including premature ejaculation, low libido, and male infertility.

DHEA

DHEA, also known as the “mother of all hormones” is a pro-hormonal building block of natural testosterone. DHEA is the most important human prohormone. It is the prohormone that converts into testosterone in men. DHEA levels decrease with age. Production peaks in a man's early 20's and declines about 10% every 10 years. Low levels of testosterone can lead to low sex drive and smaller sex organ. DHEA is the precursor for estrogen and testosterone, which have also been reported to decrease with the aging (Morales, Nolan, Nelson & Yen, 1994). Research shows that cortisol, the stress hormone is elevated in major depression. DHEA has been found to counteract cortisol.

DHEA supplements help those who have adrenal deficiency and low levels of DHEA (Araghinikam, Chung, Nelson-White, Eskelson & Watson, 1996; Yen, Morales, & Khorram, 1997). The benefits from DHEA supplements (in those who are deficient) include improved sense of vigor and wellbeing, more alertness and stamina, better memory, and enhanced sexual interest and performance.

Korean Ginseng

Korean Ginseng is classified as an adaptogen, and it is said to promote longevity, vitality, and act as aphrodisiac and sexual tonic. Research studies have found a slight connection between sex drive and consuming ginseng, although a direct link and the mechanism of actions are still unknown (Murphy & Lee, 2002). Moreover, some tests with lab animals and ginseng have shown that Korean ginseng promotes the growth of male reproductive organs, increases sperm and testosterone levels, and increases sexual activity in laboratory animals. In general, scientists believe the link between ginseng and sex drive is due to ginseng's effect of strengthening overall health and balancing the hormonal system. A caution about Ginseng is that caffeine products or other stimulants should be avoided as they may potentates the effects of Ginseng.

Tongkat Ali

Tongkat Ali (Eurycoma longifolia) has an ancient reputation as an aphrodisiac in Malaysia and Indonesia, where it's known as Pasak Bumi. Its name means “Ali's staff or walking stick” in reference to its effects on male sexuality. Tongkat Ali is highly valued to increase stamina, energy and vitality in men. The decoction is used for sexual connotations (penile firmness and strength). Tongkat Ali enhances natural testosterone and cGMP production. When optimal testosterone is achieved, sexual health and vitality are boosted. This traditional herbal plant with wide spread pro-sexual and healing properties helps to stimulate the natural defense mechanism of the body to all kinds of insults, by increasing the body's ability to protect itself. It is a clinically proven contributor to male sex drive and sexual stamina which is widely reported to heighten the urge for sex and prolong sexual activity before ejaculation. Of particular interest was the 1998 report on Tongkat Ali's ability to increase sexual motivation in sexually naive male rats published by the University Sans Malaysia (Ang 1988). The study was designed to evaluate the aphrodisiac properties of Tongkat Ali. An electric grid separated sexually inexperienced male rats from estrus-receptive females on the other side of the grid. One group of virgin male rats were treated with TongKat Ali, the other were not. Tongkat Ali caused the virgin male rats to hop across the electric grid mounting the receptive females and ejaculating more frequently than the non-treated controls.

Eurycoma Longifolia Jack

Research has shown that Eurycoma Longifolia Jack contains several phytochemicals that support healthy testosterone levels required for optimal male sexual functioning. It is also said to promote sexual health and sex drive in men, and increase mental alertness. (Ang & Sim, 1998). Results showed that repeated dosing of E. longifolia root extracts exerted pronounced stimulation of copulatory behavior in non-copulator male rats with high level of maintenance of both intromissions and ejaculations.

Yohimbine

Yohimbine has been approved by the FDA as a vasodilator and for treatment of male impotence, whether due to vascular problems, diabetes, or psychogenic causes. It is has been said that the natives of West Africa used yohimbe for centuries to help promote love as well as war. It is also believed that Africans used yohimbe during fertility ceremonies as a means of increasing libido, enhancing penis size and improving sexual performance.

Research has also demonstrated its use in the treatment of ED (Ernst & Pittler, 1998; Morales, Condra, Owen, Surridge, Fenemore & Harris, 1987; Riley, 1994; Sonda, Mazo, & Chancellor, 1990; Vogt, Brandl, Kockott, Schmitz, Weigand, Schadrack, & Gierend, 1997). Scientific studies on yohimbe conducted since the 1930s have confirmed that the herbs have definite effects on aspects of sexual performance and have confirmed its ability to increase blood flow to the penis and cause erectile stimulation and penile size enhancement. “Long term usage of Yohimbe can result in erections that are firmer, larger and longer lasting than normal and encourage permanent gains in penis size”.

L-Arginine

L-Arginine is an amino acid present in the proteins of all life forms. While it is best known as a growth hormone releaser, the decrease in growth hormone in the human body with aging is a major reason why muscle mass tends to decrease with age, body fat tends to increase with age, and also accounts in part for the slower rate of skin growth with aging (Kirk, et al, 1993) which results in thinner and less flexible skin.

Nitric oxide now also appears to be the neurotransmitter responsible for converting short-term memories to long-term memories in the brain. This is another ability that often declines with aging. Most importantly, is its precursor role to nitric oxide or NO. Nitric oxide plays a very important role in sexual function. It is epithelial-derived relaxing factor, the chemical secreted by the lining of human blood vessels in the penis that causes the blood vessels to dilate, and thus determines in part the extra blood flow into the penis during the process of erection (Lerman, Bumette, Higano, et al., 1998);. Adequate nitric oxide also helps to ensure that this allocation of blood flow to the penis is accomplished without undue increase in blood pressure.

L-Arginine is included in the medically designed EXTENZE™ formula to enhance and compliment the affect of its penile blood flow enhancing ingredients and to compliment DHEA and its other longevity, circulation, and testosterone promoting sexual nutrition supplements.

Several recent studies suggest a statistical significance to improvement of sexual function associated with L-Argenine's clinical use (Barbul, (1986); Clarkson, Adams, Powe, et al., 1996); Isdori, Lo Monaco, & Cappa, 1981).

Zinc

Zinc is essential and required to for the body to manufacture optimal testosterone levels. Men with zinc deficiency may have a problem with sexuality and a shortage may induce a low sperm count, loss of sexual desire, and emotional problems. Lack of zinc may also cause swelling of the prostate gland, which will in turn slow sperm traveling up from the testes. This swelling will also slow the release of prostatic secretions. Thus, zinc has a possible role for spermatogenesis and steroid genesis. Zinc's concentration in seminal plasma should be considered as one of the factors responsible for decreased testicular function in infertile male subjects. Zinc may also be helpful in fighting prostate infection and inflammation in older men. (Ali H, et al., “Relationship of serum and seminal plasma zinc levels and serum testosterone in oligospermic and azoospermic infertile men”, J Coll Physicians Surg Pak, 2005 November;15(11):6713).

### Method

### Subject Selection

Subjects were solicited through local newspaper ads. The goal of this study was to obtain at least 25 participants of which 20 completed the eight week study (n=20). Inclusion criteria included gender (male only), between the age of 18 and 65 with a normal sexual history, no erectile dysfunction (ED) or sexual dysfunction and participants must have agreed to be reached by phone for the weekly evaluation of each measured variable. The study required a commitment of at least 8 weeks participation. Subjects were also required to meet with primary researcher in person at week one and at week eight at a private clinical setting.

Exclusion criteria included: 1) severe hypertension (whether or not pharmaceutically controlled) and 2) medically diagnosed impotence or sexual dysfunction due to physical/medical conditions. Both criteria requirements were reviewed by a medical doctor.

Participants were instructed to take one tablet of EXTENZE™ daily, at the same time each day to achieve a more controlled day to day level. Each subject received two bottles of EXTENZE™, and an availability day and time was agreed upon. To increase the likelihood of compliance with the research design requirements, a one year supply of EXTENZE™ was offered to each participant as incentive to complete study.

### Measures

Questionnaire

Subjects were given a series of 13 questions to answer each week. Criterion Validity of questions was assessed under three domains: 1) Sexual Desire/Sexual Energy/Passion, 2) Sexual Power/Pleasure/Performance, and 3) Overall Sex Life Improvement.

Limited to 7 day intervals, ratings were documented utilizing the Likert scale from one to ten. Results were obtained weekly (past seven days) through phone consultations using the same 13 questions at each time.

### A. Sexual Desire/Sexual Energy/Passion

The following four questions were considered:

1.) How many significant events have you had in the lasts week? (i.e., vaginal intercourse, anal intercourse, masturbation, oral sex,)

2.) How many times did you masturbate in the last week?

3.) How many times did you become hard or sexually aroused by erotic content in the last week? (Including all forms of media, internet, personal encounters)

4.) How often did you sexually fantasize last week?

### B. Sexual Power/Pleasure/Performance

The following six questions were considered under heading two:

5.) Was your confidence and ability to achieve and maintain a hard erection improved?

6.) Did it take less stimulation to achieve a hard erection?

7.) Was your erection firmer, stronger, longer or harder?

8.) How pleased were you with the improvement in your erection?

9.) Were you able to maintain a firm, hard erection longer during sex?

10.) Was the pleasure you got from your penis more than you previously experienced?

### C. Overall Sex Life Improvement

The following three questions were considered:

11.) Were you more pleased with your sexual performance this week than last?

12.) Were you more pleased with your sex life as a whole this week than last?

13.) Has taking EXTENZE™ (described in the study as “the pill”) contributed to an overall improvement in the quality of your sex life?

There was a 99% improvement in sexual performance satisfaction after taking EXTENZE™ for eight weeks. These results provide evidence to conclude that when taking EXTENZE™ it is advisable to continue taking the pill for at least eight weeks before the full effect can be noted. Analysis did not show a statistically significant improvement between the first and the fourth week of taking EXTENZE™, but did show a highly significant improvement in increased sexual pleasure and performance by the time study participants had taken EXTENZE™ for eight weeks.

Penile Measurements

Penile measurements were also documented at intervals of week one, week two, month one and month two. The study showed up to 50% improvement in flaccid and erect length. During the first interview, participants were asked to purchase a measuring tape, and the method of measurements was explained in detail to the participants. The demonstration of measurement requirements and procedure was then reviewed in private with each subject. Inconsistency of measurement could not be controlled using this design protocol method due to personal reasons (i.e. poor or inconsistent measuring techniques, shyness, lack of self-esteem, ego, size inadequacy, etc.) Despite these limitations, significant penile size enhancement length and girth was observed in 50% and 45% respectively. Future study might best be obtained by the direct measurement of penile size during nocturnal tumescence (nighttime sleep erections). This can and has been measured by the use of penile RigiScan computer monitors which attach to the base, mid part, and tip of the relaxed penis with Velcro and connect to a thigh attached microprocessor (similar to the Holter monitor for the heart) that records penile blood flow during sleep, thereby allowing clinical observation (6). RigiScan penile tumescence monitors have been used by scientists and doctors for decades to reliably document frequency and degree of penile erection.

### Results

### Analysis of Data

A total of 32 subjects responded to the request for participation through local newspaper ads. Of the initial subject pool, 20 completed the eight week study (n=20).

Results were obtained weekly (past seven days) through phone conversations using the same 13 questions at each time. The questionnaire was comprised of 13 questions, which in turn were divided under three Domains: Sexual Desire/Sexual Energy/Passion, Sexual Power/Pleasure/Performance, and Overall Sex Life Improvement. Each question was rated by the participants on a Likert scale from one through ten.

All statistical analysis was performed on raw data by Dr. Brian Garman, Professor and Chairman, Department of Mathematics, University of Tampa. The psycho-somatic effect of taking such sexual enhancement drugs was taken into consideration and during questioning attention remained focused on each participant's perception of improvement.

It is worth mentioning that subjects were asked to report penile measurements in either flaccid or erect state at week one, four and eight. However, as previously noted in the subject design section, the penile measurement data were not submitted for analysis due to subject's reluctance to comply with the standardized measurement procedure as outlined in the protocol. Although “self measurement” seemed to be a rational alternative, “self” measurements were not repeatable or consistently controlled. Despite this, a remarkable 50% size increase in erect length and 30% girth improvement was observed.

The study showed that the results improved each week after beginning EXTENZE™. This weekly improvement in sexual enhancement continued throughout the entire eight week study period.

The results for each of the thirteen questions had staggering similarities. Focus will be on one question only of each variable group under each heading. A p-value <0.01 is called highly significant. Week one was the first interview before the start of EXTENZE™.

Domain One: Sexual Desire/Sexual Energy/Passion. The overall p-value for question four under this heading in number of sexual fantasies is p=0.0016 and therefore highly significant. The results show a 99% level of difference between week one and week eight utilizing a Fisher post-hoc paired t-test to see which means were different. The occurrence of sexual fantasies rose significantly in all participants taking EXTENZE™ once a day.

Domain Two: Sexual Power/Pleasure/Performance was examined by six questions. The p-value for question one evaluating confidence to achieve erection level was p=0.0498. (p<0.05 is called significant) The post hoc shows a 95% level of significant difference between week one and week eight in the confidence of the participants to be able to achieve an erection, which in turn increases the number of sexual events.

Domain Three: Overall Sex Life Improvement. The p-value for question one evaluating satisfaction with overall sexual performance between week one and week eight was p=0.0015 and therefore highly significant. The post-hoc analysis shows a 99% improvement in satisfaction with sexual performance after taking EXTENZE™ for eight weeks.

The differences between week one and eight are of such significance that a similar study, with control group, is warranted. These results delivered enough evidence to conclude that when taking EXTENZE™ it is advisable to continue for at least eight weeks before the full effect can be noted. Results of this study suggest a positive influence and increased sexual enhancement and pleasure. Post-hoc t-test did not show a significant difference between week one and week four.

### Interpretation of Results

The following are the results on a percentage basis:

| | |
|---|---|
| Increase in weekly sexual events. | 18/20 = 90% |
| (Intercourse, masturbation etc.) | |
| Increase in sexual desire | 17/20 = 85% |
| Increase in sexual fantasies | 20/20 = 100% |
| Improvement in confidence to | 15/20 = 75% |
| achieve an | |
| Erection | |
| Increase in penile sensitivity, less | 13/20 = 65% |
| stimulation needed | |
| Increase in firmness of erections | 18/20 = 95% |
| Increase in patient satisfaction with | 18/20 = 90% |
| Erections | |
| Increase in maintaining erection to | 20/20 = 100% |
| complete intercourse | |
| Increase in overall pleasure | 15/20 = 75% |
| Increase in personal sexual | 17/20 = 85% |
| performance | |
| Satisfaction | |
| Increase in satisfaction of sex life as a | 16/20 = 80% |
| Whole | |
| Increase in overall improvement | 20/20 = 100% |
| including | |
| sexual activity, vim, vigor, | |
| performance | |

### Conclusions

Results of this study suggest a positive influence and increased sexual enhancement and pleasure. The differences between week one and eight are of such significance that a similar study, with control group, is warranted. These results delivered enough evidence to conclude that when taking EXTENZE™ it is advisable to continue for at least eight weeks before the full effect can be noted.

What is claimed is:

1. A method of increasing sexual wellness in a healthy human subject, the method comprising administering to said subject a non-toxic, effective amount of a composition comprising one or more sex steroid prohormones, or a pharmaceutically acceptable salt or metabolite thereof, wherein said composition does not elicit undesired side effects when administered to said subject.

2. The method of claim 1 wherein said sex steroid prohormone is DHEA, pregnenolone, androstenediol, or androstenedione.

3. The method of claim 1, wherein said prohormone is two or more of dehydroepiandrosterone (DHEA), pregnenolone, androstenedione, or anderostendiol.

4. The method of claim 3, wherein said composition comprises DHEA and pregnenolone, or pharmaceutically acceptable salts or metabolites thereof.

5. The method of claim 1, wherein administering said composition does not produce androgen-associated anabolic side effects in said subject.

6. The method of claim 1, wherein said healthy subject does not have hypoactive desire, hypoactive arousal, orgasmic dysfunction, erectile dysfunction, impotence, or mental illness.

7. The method of claim 1, wherein said composition is present in therapeutically effective amounts to increase effects of testosterone effect when administered to a healthy subject.

8. The method of claim 1, wherein said composition is administered for a time sufficient to improve satisfaction with overall sex life in said subject.

9. The method of claim 8, wherein said increased testosterone function enhances the level of sexual wellness by an end of a period of time.

10. The method of claim 1, wherein increased sexual wellness includes an increase in sexual desire, arousal, response, or performance.

11. The method of claim 1, wherein increased sexual wellness includes an increase in sexual desire, sexual arousal, sexual response, or sexual performance.

12. The method of claim 1, wherein increased sexual wellness includes an increase in sexual passion.

13. The method of claim 1, wherein said increased wellness includes an improved quality of orgasm.

14. The method of claim 1, wherein said subject is a female.

15. The method of claim 14, wherein said composition is administered for a time sufficient to improve clitoral sensation during sexual stimulation.

16. The method of claim 14, wherein said composition comprises DHEA and pregnenolone, or pharmaceutically acceptable salts or metabolites thereof.

17. The method of claim 16, wherein said pregnenolone is present at 100-200 mg per unit dose.

18. The method of claim 14, wherein said DHEA is present at 25 mg-100 mg per unit dose.

19. The method of claim 1, wherein said subject is a male.

20. The method of claim 19, wherein said composition comprises DHEA and pregnenolone, or pharmaceutically acceptable salts or metabolites thereof.

21. The method of claim 20, wherein said pregnenolone is present at 10-200 mg per unit dose.

22. The method of claim 20, wherein said DHEA is present at 100 mg per unit dose.

23. The method of claim 2, wherein DHEA comprises DHEA sulfate (DHEAS) or an ester thereof.

24. The method of claim 2, wherein DHEA comprises a metabolite of DHEA.

25. The method of claim 24, wherein said DHEA metabolite is 7-keto dehydroepiandrosterone (7-keto DHEA).

26. The method of claim 1, wherein said composition is administered to said subject in a route selected from the group consisting of oral, topical, transdermal, intranasal, sublingual, buccal, and transrectal delivery.

27. The method of claim 1, wherein said composition is provided in a unit dosage form.

28. The method of claim 28, wherein said unit dosage form is in the form of a capsule or a tablet.

29. The method of claim 1, wherein said composition further comprises a sexual nutrient, wherein said composition is optimized to enhance sexual wellness in said subject.

30. The method of claim 29, wherein said sexual nutrient is yohimbe, maca, Tribulus terrestris, ginseng, horny goat weed or damiana.

31. A method of maintaining a desired level of sexual wellness in a healthy subject, the method comprising
assessing sexual wellness in a healthy subject;
administering to said subject a non-toxic, effective amount of a composition comprising a prohormone, or a pharmaceutically acceptable salt or metabolite thereof, wherein said composition does not elicit undesired side effects in said subject,
evaluating sexual wellness following administration of said composition to said subject; and
readministering said composition to said subject as needed to maintain a desired level of sexual wellness.

31. The method of claim 30, wherein said prohormone is dehydroepiandrosterone (DHEA), pregnenolone, androstenedione or anderostendiol.

32. The method of claim 31, wherein both DHEA, pregnenolone, androstenedione oranderostendiol are administered to said subject.

33. The method of claim 31, wherein the readministered dose of DHEA, pregnenolone androstenedione, or anderostendiol is varied depending on the evaluated sexual wellness of said subject.

34. The method of claim 30, wherein the administering includes initially administering an elevated dosage of the composition to attain the enhanced level of sexual wellness by the end of a period of time and thereafter administering a dosage of the composition daily that contains less of the composition than the elevated dosage and still provides the enhanced level of sexual wellness.

35. A nutritional supplement for enhancing sexual wellness in a subject, comprising ingredients that include a combination of
dehydroepiandrosterone (DHEA) and
pregnenolone in an amount effective to increase sexual wellness in a healthy human subject.

36. A method of making a composition for enhancing a level of sexual wellness in a subject, the method comprising combining dehydroepiandrosterone (DHEA) and pregnenolone in an amount effective to increase sexual wellness when administered to a healthy human subject.
